(19) United States
(12) Patent Application Publication
(10) Pub. No.: US 2020/0337960 A1
(43) Pub. Date: Oct. 29, 2020

(54) FILM WITH A COLOURED METALLIC EFFECT TO BE APPLIED AS MAKEUP

(71) Applicant: ELEGANT BEST INVESTMENT LIMITED, Hong Kong (CN)

(72) Inventors: Lise Masson, Launaguet (FR); Pauline Saurel, Saint Orens de Gameville (US); Xiaodong He, Auzeville Tolosane (FR); Julie Saintecatherine, L'Union (FR)

(73) Assignee: ELEGANT BEST INVESTMENT LIMITED, Hong Kong (CN)

(21) Appl. No.: 16/760,320

(22) PCT Filed: Nov. 7, 2018
(86) PCT No.: PCT/IB2018/001242

§ 371 Date: Apr. 29, 2020

(30) Foreign Application Priority Data
Nov. 8, 2017 (CN) 201711091320.2
Nov. 8, 2017 (FR) 1760461

(51) Int. Cl. A61K 8/29; A61K 8/02; A61Q 1/10; A61K 8/81

(52) U.S. Cl. CPC A61K 8/29; A61K 8/0241; A61Q 1/10; A61K 2800/621; A61K 2800/436; A61K 2800/884; A61K 8/8152

(57) ABSTRACT

The invention relates to a skin cosmetic makeup set characterized in that it comprises:
a first composition to be applied to the skin so as to form at least one layer, called first layer; and
a second composition to be applied over all or a part of said first layer;
said first composition comprising an aqueous solvent, at least one black pigment, and at least one film-forming agent, the at least one film-forming agent representing in the range 10% to 45 % by weight of the total weight of said first composition;
said second composition comprising at least one interference pigment, the interference pigment comprising a transparent substrate coated with at least one layer of coating material, said coating material being TiO₂.

### 1. TECHNICAL FIELD OF THE INVENTION

The invention relates to a first cosmetic makeup composition, to a makeup set comprising the first composition and a second composition, to a multilayer makeup product, and to a makeup kit.

### 2. TECHNOLOGICAL BACKGROUND

Makeup compositions are colored using colorants or dyes such as soluble dyes, pigments based on metal oxides, such as iron oxides, or indeed effect pigments that produce satiny, pearlescent, metallic, or glossy effects or finishes, such as interference pigments, e.g. micas coated with metal oxides such as titanium oxide, that produce satiny, pearlescent, metallic, and glossy effects.

Such pigments are visually attractive. They reflect, diffuse, and absorb various wavelengths of incident light. Each color has its own wavelength that can be discerned by the human eye. The eyes perceive reflected light but their perception can be affected by transmitted light.

The effects of such pearlescent pigments are generally inherent to their primary substrates that have specific properties as a function of their chemical natures, of their thicknesses, and of their refractive indices. The substrates used by the cosmetics industry include mica, synthetic mica, silica, aluminum, borosilicate glass, and polyethylene terephthalate (PET). Those substrates are coated with metal oxides having high refractive indices. The optical effect that is obtained is based on an interference phenomenon that is in turn based on the difference in refractive index between the air, the coating, and the substrate. On application, the interference colors are dependent on the thickness and on the coating. The size of the substrate also has an influence on the perceived effect. Effect pigments of small size give a satiny or silky effect, whereas those of large size give a glittery effect. More complex materials such as nacres procuring a “color travel” effect are formed of structures made up of layers of different thicknesses that are responsible for the variation in color as a function of viewing angle.

Such interference pigments may be colored or colorless depending on the metal oxide used for the coating. Colorless interference pigments give a very advantageous color effect since their transparency makes it possible to give depth and naturalness. However, a drawback of such colorless interference pigments is that their transparency does not enable them to obtain a covering makeup. In addition, although the colored effect of interference pigments is particularly advantageous, interference colors are of low intensity naturally.

In order to mitigate that problem, absorption pigments such as iron oxides or carbon black have been associated with interference pigments during formulation. However, the interference pigments are then covered with the absorbent and non-transparent pigments, and therefore stand out less.

In order to intensify the interference color of interference pigments, it is also possible to associate them with organic dyes or lacquers thereof, which have very bright colors. But organic dyes and their lacquers often have intrinsic stability problems or destabilize the formulae due to their physical and chemical properties. The cosmetics regulations of the various countries also limit the number of dyes that can be used, in particular for the eye contour area, mainly for reasons of irritation and of toxicity of the organic dyes.

### 3. OBJECTS OF THE INVENTION

An object of the invention is to mitigate at least some of the known drawbacks of those types of colorless interference pigment, such as their lack of coverage, and their lack of intensity or brightness.

An object of the invention is also to provide makeup that is of colored metallic appearance, in at least one embodiment of the invention.

An object of the invention is also to provide makeup that is easy to apply, natural and comfortable, in at least one embodiment.

The invention makes it possible to obtain colors that are varied, bright, intense, glossy and metallic, without using undesirable organic dyes.

### 4. SUMMARY OF THE INVENTION

To this end, the invention provides a skin cosmetic makeup composition, called first composition, comprising an aqueous solvent, at least one black pigment, and at least one film-forming agent, the at least one film-forming agent representing in the range 10% to 45% by weight of the total weight of said first composition.

Indeed, the inventors have observed that said first composition of the invention offers an excellent base on which to apply a second makeup composition based on colorless interference pigments. In addition to the durability of adhesion and to the coverage procured by this base, it also, surprisingly, imparts a very intense metallic appearance to the second makeup composition based on colorless interference pigments.

Thus, the invention also provides a skin cosmetic makeup set comprising:
a first composition to be applied to the skin so as to form at least one layer, called first layer; and
a second composition to be applied over all or a part of said first layer;

said first composition comprising:
an aqueous solvent;
at least one black pigment; and
at least one film-forming agent, the at least one film-forming agent representing in the range 10% to 45% by weight of the total weight of said first composition;

said second composition comprising at east one interference pigment, the interference pigment comprising a transparent substrate coated with at least one layer of “coating material”, said coating material being titanium dioxide (TiO₂).

### 5. DETAILED DESCRIPTION

First Composition

The cosmetic makeup composition, called first composition, according to the invention is to be applied to the skin, and preferably to the upper and/or lower eyelids. Preferably, the first composition is to be used as a makeup base.

The first composition according to the invention comprises the at least one film-forming agent in the range 10% to 45% by weight of the total weight of the first composition, preferably in the range 15% to 30% by weight of the total weight of the first composition, and more preferably in the range 20% to 30% by weight of the total weight of the first composition. The high concentration of film-forming agent makes it possible to obtain a first layer that is uniform. This high concentration of film-forming agent combined with the presence of black pigment that absorbs light results in the first composition having light absorption that is very high. This capacity of the first composition to absorb light makes it possible to obtain a metallic effect with very high intensity when a second composition comprising interference pigments is spread over said first composition.

The light absorption capacity of the first composition is measured by the lightness (L) in the CIELAB color space. In a preferred embodiment, the lightness of the first composition is less than 27.5, preferably less than 25, more preferably less than 20, and even more preferably less than 15.

Measuring lightness in the CIELAB space (more particularly the CIELAB76 space in the sense of the International Commission on Illumination (CIE) in accordance with Recommendation 15: 2004) is a technique that is usual for the person skilled in the art. To do so, the person skilled in the art may use any type of colorimeter or any type of spectrophotometer, e.g. a Colorcatch Nano instrument. Lightness may be measured after the first composition has been spread on a plate of artificial skin, such as, for example the BIOSKIN PLATE 10 EUROPE FACE by Beaulax Co. Ltd. or on the white zone of a contrast card, e.g. Chart 2810, sold by the company BYK.

The light absorption capacity of the first composition is also characterized by opacity. Opacity is measured by measuring the lightness (L) over the black zone and over the white zone of a contrast card on which the composition is preferably spread by means of a drawdown bar, e.g. the one referenced 1222782 and sold by BYK. Opacity is calculated by dividing the lightness over the black zone (L_{black zone}) by the lightness over the white zone (L_{white zone}), multiplied by 100 (Opacity (%)=(L_{black zone}/L_{white zone})×100). Opacity of 100% indicates that coverage is full. In one embodiment, the opacity of the first composition is greater than 75%, preferably greater than 80%, and more preferably greater than 85%.

In addition, the first composition has high adhesion enabling the second composition to adhere securely and durably to the first composition. To achieve this, a film-forming agent having good adhesion is preferred. Since adhesion is generally correlated to a low glass transition temperature (Tg), film-forming agents having low glass transition temperatures are therefore preferred. For example, the film-forming agent may have a glass transition temperature less than 20° C., preferably less than 10° C., more preferably less than 0° C., and even more preferably less than −10° C.

The at least one film-forming agent of said first composition may be selected from the group consisting of polyurethane, polyester, poly(ester-amide), polyimide, polyurea, vinyl polymer or their derivatives, acetyltributylcitrate, acrylate polymer or their derivatives, acrylamides polymer or of their derivatives, cellulose or its derivatives, chitosan or its derivatives, rosin, resin, gum, polyquaternium and of their mixtures.

The at least one film-forming agent of said first composition is preferably selected from the group consisting of polyurethane, acetyltributylcitrate, acryalates polymer or their derivatives, acrylamides polymer or their derivatives, of cellulose or its derivatives, chitosan or its derivatives, rosin, resin, gum, polyquaternium and their mixtures, more preferably selected from the group consisting of an acrylates polymer and/or an acrylates polymer mixture, and even more preferably selected from the group consisting of a mixture of acrylates copolymer and of acrylate(s) copolymer and of ethylhexyl acrylate.

Advantageously, the aqueous solvent of said first composition represents in the range 1% to 89.5% by weight of the total weight of said first composition, and preferably in the range 50% to 85%. The solvent is preferably water.

The black pigment that is intended to absorb light may be any black pigment that is usable in cosmetics. For example, the black pigment may be a metal oxide and/or carbon black. Advantageously, the black pigment represents in the range 0.5% to 30% by weight of the total weight of said first composition, and preferably in the range 1% to 20%, in the range 1% to 5%, or in the range 1% to 4% by weight of the total weight of said first composition. Due to its back color, the black pigment enables the composition to absorb all of the light transmitted through the second composition, and thus to obtain a maximum effect of the interference color reflected in the various layers of the interference pigment.

Preferably, said first composition does not contain any pigment other than the black pigment(s); in particular, it does not contain any interference pigments.

Finally, said first composition may further comprise at least one gelling agent, at least one humectant, at least one fragrance, at least one preservative, and/or any other active ingredient that is usual in cosmetics.

The at least one gelling agent may be a natural gum, preferably chosen from the group made up of guar gum, of locust bean gum and of xanthan gum, and/or an organophilic clay, preferably selected from the group consisting of stearalkonium hectorite and of Bentone.

Preferably, the first composition is peelable. A composition that is “peelable” means that once applied to the skin, the composition can be detached therefrom by being peeled off, without using any makeup remover.

Second Composition

The present invention also relates to a second composition.

Said second composition comprises at least one interference pigment. The interference pigment comprises a transparent substrate, e.g. mica, coated with at least one layer of a material, referred to as a “coating material”, the coating material being titanium dioxide (TiO₂). The coating material and the thicknesses are chosen in such a manner that a color is produced by interferences.

The second composition is to be applied over all or some of the first composition, thereby forming a very intense metallic effect.

Said second composition may be in “stick” form or in powder form, and is preferably in powder form, e.g. in loose powder form or in baked powder form.

In addition to the at least one interference pigment, the second composition may further comprise a cosmetically acceptable excipient. It may also further comprise fragrances, preservatives, and/or any other active ingredient that is usual in cosmetics.

Preferably, the at least one interference pigment is a multilayer interference pigment. The expression “multilayer interference pigment” means a pigment capable of producing a color by a phenomenon of interference between light rays reflected by a plurality of superposed layers that have different refractive indices, in particular a succession of high refractive index and low refractive index layers.

The second composition may comprises a single interference pigment or a plurality of interference pigments.

The substrate of the interference pigment may be coated with one or more layers of material(s) on one or more faces.

The at least one interference pigment of the second composition may comprise a transparent substrate selected from the group consisting of silica, mica and glass.

The substrate may have a flat shape or some other shape.

The substrate may comprise natural mica, synthetic mica, glass, or silica. The nature of the substrate may be chosen as a function of the desired glossiness. For example, for a very glossy rendering, a glass substrate may be preferred.

For example, the size of the particles of the interference pigment, given by the mean particle-size at half of the population, or “DSO”, lies in the range 1 micrometer (μm) to 2000 μm, or even better in the range 5 μm to 1000 μm.

Said at least one interference pigment represents in the range 1% to 99% by weight of the total weight of said second composition, and preferably in the range 15% to 50% by weight of the total weight of said second composition.

The interference pigment may also be a goniochromatic pigment. For example, the goniochromatic pigment may be chosen from among interference multilayer structures.

As a goniochromatic pigment, it is also possible to use certain nacres, effect pigments on synthetic substrates, in particular substrates of the silica or borosilicate type, or interference flakes coming from a polyterephthalate film.

Cosmetic Makeup Set

The present invention also relates to a skin cosmetic makeup set comprising:
said first composition according to the invention, said first composition being to be applied to the skin so as to form at least one first layer; and
said second composition according to the invention, said second composition being to be applied over all or a part of said first layer.

Typically, the skin cosmetic makeup set of the invention comprises:
a first composition to be applied to the skin so as to form at least one first layer; and
a second composition to be applied over all or a part of said firstayer.

said first composition comprising:
an aqueous solvent;
at least one black pigment; and
at least one film-forming agent, the at least one film-forming agent representing in the range 10% to 45% by weight of the total weight of said first composition;

said second composition comprising at least one interference pigment, the interference pigment comprising a transparent substrate coated with at least one layer of “coating material”, said coating material being TiO₂.

The first and second compositions of the cosmetic set of the invention are the compositions that are described in the preceding paragraphs.

Thus, the first composition of the cosmetic set preferably has lightness (L) in the CIELAB space that is less than 27.5. Advantageously, the first composition is to be used as a makeup base. The at least one film-forming agent of the first composition is selected from the group consisting of acetyltributylcitrate, acrylate polymer or their derivatives, acrylamide polymer or their derivatives, cellulose or its derivatives, chitosan or its derivatives, rosin, resin, gum, polyquaternium and their mixtures. In a preferred embodiment, the at least one film-forming agent of said first composition represents in the range 15% to 30% by weight of the total weight of said first composition. Also in a preferred embodiment, the black pigment of said first composition represents in the range 0.5 to 30% by weight of the total weight of said first composition.

Advantageously, a single layer of the first composition is applied.

On or more layers of said second composition may be applied over all or some of said first layer. However, it is preferable to apply only a single layer of the second composition over the first composition.

In addition, it is also preferable for the second composition to be applied over all of said first layer.

In a preferred embodiment, the set of the invention is peelable.

Multilayer Cosmetic Makeup Product

The present invention also relates to a skin cosmetic makeup product comprising:
at least one first layer of said first composition of the invention; and
at least one second layer of said second composition of the invention, said second layer being applied over all or a part of said first layer.

Typically, the first and second compositions of the cosmetic product form two distinct layers that are sticked together.

Preferably, the multilayer product of the invention is peelable.

Cosmetic Makeup Kit

The present invention also relates to a skin cosmetic makeup kit comprising:
said first composition of the invention; and
said second composition of the invention.

Makeup Method

Finally, the invention provides a skin makeup method comprising:
applying at least one first layer of said first composition of the invention to the skin; and
then applying over all or a part of said first composition at least one layer, called second layer, of the second composition of the invention.

The invention is also illustrated by the following figures and examples. However, these examples and figures should not be interpreted as limiting the scope of the present invention.

### 6. FIGURES

FIGS. 1 and 2 diagrammatically show in simplified manner the absorbance effect that the lower layer of a multilayer cosmetic product can have on the light rays.

FIG. 1 is a diagrammatic view of the interaction between an incident light ray and a multilayer product comprising an interference pigment and a black absorbent lower layer.

FIG. 2 is a diagrammatic view of the interaction between an incident light ray and a multilayer product comprising an interference pigment and a white lower layer.

When an incident light ray 1 comes into contact with an interference pigment 2, a fraction of the ray (ray 1) is reflected (ray 4) and the remaining fraction (ray 5) is refracted through the interference pigment.

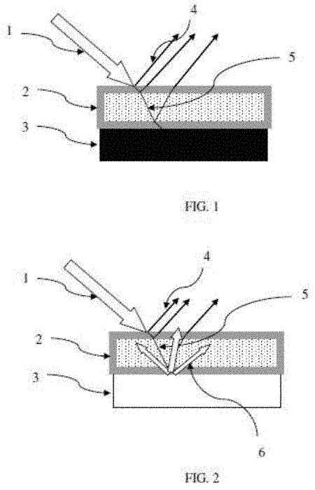

If the lower layer 3 is black (FIG. 1), then the ray 5 transmitted through the interference pigment is absorbed by the black layer.

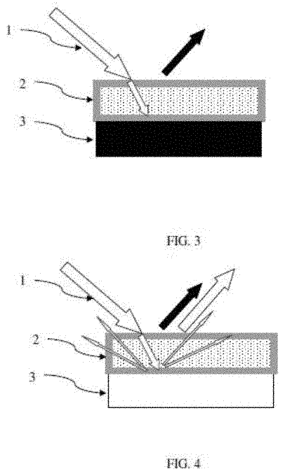

If the lower layer 3 is white (FIG. 2), then the ray 5 transmitted through the interference pigment is diffused and reflected by the white layer.

Thus, when the lower layer diffuses light, the perception of the interference color is lower because the eye also receives the diffused or reflected light (FIG. 4). Whereas, when the lower layer is absorbent, the perceived intensity or brightness reflected by the interference pigment is relatively much greater (FIG. 3).

### 7. EXAMPLES

### Preparation Examples: Eyeliner Having a Colored Metallic Effect

First Composition:

Add the ingredients according to the percentage indicated, and mix.

Example 1A:

| | |
|---|---|
| Mixture of acrylates copolymer and of copolymer of | 25% |
| acrylates and of ethylhexyl acrylate | |
| Carbon black | 3% |
| Water | 72% |

Comparative example 1B:

| | |
|---|---|
| Mixture of acrylates copolymer and of copolymer of | 25% |
| acrylates and of ethylhexyl acrylate | |
| Titanium dioxide | 3% |
| Water | 72% |

Comparative example 1C:

| | |
|---|---|
| Mixture of acrylates copolymer and of copolymer of | 25% |
| acrylates and of ethylhexyl acrylate | |
| Water | 75% |

Example 1F:

| | |
|---|---|
| Mixture of acrylates copolymer and of copolymer of | 25% |
| acrylates and of ethylhexyl acrylate | |
| Carbon black | 1% |
| Water | 74% |

Example 1G:

| | |
|---|---|
| Mixture of acrylates copolymer and of copolymer of | 25% |
| acrylates and of ethylhexyl acrylate | |
| Carbon black | 4% |
| Water | 71% |

Comparative example 1H:

| | |
|---|---|
| Mixture of acrylates copolymer and of copolymer of | 12.5% |
| acrylates and of ethylhexyl acrylate | |
| Carbon black | 3% |
| Water | 84.5% |

Second Composition:

Add the ingredients according to the percentage indicated, and mix.

Example 2-2:

| | |
|---|---|
| SUNSHINE ® Spectral Red (Titanium Dioxide (and) Synthetic | 30% |
| Fluorphlogopite, SunChemical) | |
| Excipients | 70% |

Example 2-3:

| | |
|---|---|
| SUNSHINE ® Northern Lights (Titanium Dioxide (and) Synthetic | 30% |
| Fluorphlogopite (and) Magnesium Silicate, SunChemical) | |
| Excipients | 70% |

### Comparative Example I

Add the ingredients according to the percentage indicated, and mix.

| | |
|---|---|
| Mixture of acrylates copolymer and of acrylates copolymer | 25% |
| and of ethylhexyl acrylate | |
| Carbon black | 3% |
| SUNSHINE ® Northern Lights | 30% |
| Water | 42% |

LAB Measurement on Artificial Skin

L*a*b measurements in the CIELAB space were taken with the COLORCATCH NANO instrument and the RAL Icolours application. The samples of composition 1 were spread using the drawdown bar referenced 1222782 and sold by the company BYK, via the side having 6-mil, over an artificial skin sold by the company BEAULAX, CO., LTD, reference BIO SKIN PLATE 10 EUROPE FACE.

Impact of the Color of the First Layer:

Comparison of the first composition containing a black pigment, or a white pigment, or no pigment, before and then after application of the second composition containing the nacre SUNSHINE® Spectral Red or the nacre SUNSHINE® Northern Lights:

| | | L | a | b |
|---|---|---|---|---|
| A1 | Composition 1A (black pigment) | 13.6 | 5.4 | −3.1 |
| A2 | Composition 1A (black pigment) + | | | |
| | Composition 2-2 (SUNSHINE ® Spectral | 56.3 | 39.6 | −13.1 |
| | Red) | | | |
| A3 | Composition 1a (black pigment) + | 76 | −29.4 | 8.7 |
| | Composition 2-3 (SUNSHINE ® Northern | | | |
| | Lights) | | | |
| B1 | Composition 1B (white pigment) | 96.1 | −0.1 | 0.1 |
| B2 | Composition 1B (white pigment) + | 96.1 | 0.8 | 0.2 |
| | Composition 2-2 (SUNSHINE ® Spectral | | | |
| | Red) | | | |
| B3 | Composition 1B (white pigment) + | 97.4 | −0.2 | −0.1 |
| | Composition 2-3 (SUNSHINE ® Northern | | | |
| | Lights) | | | |
| C1 | Composition 1C (without pigment) | 71.9 | 13 | 19.8 |
| C2 | Composition 1C (without pigment) + | 77.4 | 19.8 | 5.3 |
| | Composition 2-2 (SUNSHINE ® Spectral | | | |
| | Red) | | | |
| C3 | Composition 1C without pigment) + | 78.1 | 4.9 | 13.5 |
| | Composition 2-3 (SUNSHINE ® Northern | | | |
| | Lights) | | | |
| D1 | Bare skin | 67.8 | 13.4 | 22.2 |
| D2 | Bare skin + Composition 2-2 | 71.6 | 13.6 | 14.9 |
| | (SUNSHINE ® Spectral Red) | | | |
| D3 | Bare skin + Composition 2-3 | 75.5 | 5.8 | 12.7 |
| | (SUNSHINE ® Northern Lights) | | | |
| E1 | Ombre éclat lumière Noir* -Nocibé | 28.6 | 8.2 | 7.3 |
| E2 | Ombre éclat lumière Noir* -Nocibé + | 43.5 | −8.7 | 5.0 |
| | Composition 2-2 (SUNSHINE ® Spectral | | | |
| | Red) | | | |
| E3 | Ombre éclat lumière Noir* -Nocibé + | 51 | 13.4 | −0.1 |
| | Composition 2-3 (SUNSHINE ® Northern | | | |
| | Lights) | | | |

*Ombre éclat lumière Noir: Black eyeshadow by the company Nocibé

Conclusions:
With the white pigment, (B2 & B3), the values for a and b were close to 0. The interference color of the nacre was not visible.
Without pigment, test C2 showed an increase in red (a), and a decrease in yellow, relative to the color of the skin (C1, D1). Test C3 showed a decrease in red (a), and a decrease in yellow (b), relative to the color of the skin (C1, D1). The interference color of the nacres could thus be seen in part on the skin, without a colored background base. However, if the values a and b obtained for these tests C2 and C3 are compared with the values obtained on a black background base (A2, A3), it can be seen clearly that they are much closer to the color of the skin and much less intense.
Tests E2 and E3, where the second composition was applied over commercially available black eyeshadow, show a and b values close to zero, and much less intense than those obtained on composition 1A.
Only the black pigment associated with the film-forming agents made it possible to reveal the intense red color (a=39.6) of the mother-of-pearl SUNSHINE® Spectral Red (A2), and the intense green color (b=−29.4) of the nacre SUNSHINE® Northern Lights.

Impact of the Percentage of Black Pigment:

Comparison of the first composition containing a different percentage of black pigment before and then after application of the second composition containing the nacre SUNSHINE® Spectral Red or the nacre SUNSHINE® Northern Lights:

| | | L | a | b |
|---|---|---|---|---|
| A1 | Composition 1A (25% film-forming | 13.6 | 5.4 | −3.1 |
| | polymers, 3% black pigment) | | | |
| A2 | Composition 1A (25% film-forming | 56.3 | 39.6 | −13.1 |
| | polymers, 3% black pigment) + | | | |
| | Composition 2-2 (SUNSHINE ® Spectral Red) | | | |
| A3 | Composition 1A (25% film-forming | 76 | −29.4 | 8.7 |
| | polymers, 3% black pigment) + | | | |
| | Composition 2-3 (SUNSHINE ® Northern | | | |
| | Lights.) | | | |
| F1 | Composition 1F (25% film-forming | 25.3 | 7 | −0.5 |
| | polymers, 1% black pigment) | | | |
| F2 | Composition 1F (25% film-forming | 50.4 | 26.4 | −12.7 |
| | polymers, 1% black pigment) + | | | |
| | Composition 2-2 (SUNSHINE ® Spectral Red) | | | |
| F3 | Composition 1F (25% film-forming | 71.6 | −25.6 | 8 .3 |
| | polymers, 1% black pigment) + | | | |
| | Composition 2-3 (SUNSHINE ® Northern | | | |
| | Lights) | | | |
| G1 | Composition 1G (25% film-forming | 11.1 | 8.8 | −5.1 |
| | polymers, 4% black pigment) | | | |
| G2 | Composition 1G (25% film-forming | 52.8 | 32.8 | −9.5 |
| | polymers, 4% black pigment) + | | | |
| | Composition 2-2 (SUNSHINE ® Spectral Red) | | | |
| G3 | Composition 1G (25% film-forming | 76.7 | −28.5 | 9.4 |
| | polymers, 4% black pigment) + | | | |
| | Composition 2-3 (SUNSHINE ® Northern | | | |
| | Lights) | | | |

Conclusions:
The difference between the result obtained for 3% of black pigment and 4% of black pigment in the first composition was small. It would appear that a saturation effect is reached above 3% of black pigment.
The lightness of the first composition+the second composition was higher for A2 and A3 containing 3% of black pigment than for F2 and F3 containing 1% of black pigment. The glossiness was thus higher.
The red interference color of the nacre SUNSHINE@ Spectral Red was more intense with the first composition at 3% of black pigment than with the first composition at 1% of black pigment (a=39.6 for sample A2 and a=26.4 for sample F2).
The green interference color of the nacre SUNSHINE@ Northern Lights was more intense with the first composition at 3% of black pigment than with the first composition at 1% of black pigment (a=−29.4 for sample A3 and a=−25.6 for sample F3).

### Impact of the Percentage of Film-Forming Agents:

Comparison of the first composition containing a different percentage of fim-forming polymers before and then after application of the second composition containing the nacre SUNSHINE® Spectral Red or the nacre SUNSHINE® Northern Lights:

| | | L | A | b |
|---|---|---|---|---|
| A1 | Composition 1A (25% film-forming | 13.6 | 5.4 | −3.1 |
| | polymers, 3% black pigment) | | | |
| A2 | Composition 1A (25% film-forming | 56.3 | 39.6 | −13.1 |
| | polymers, 3% black pigment) + | | | |
| | Composition 2-2 (SUNSHINE ® Spectral Red) | | | |
| A3 | Composition 1A (25% film-forming | 76 | −29.4 | 8.7 |
| | polymers, 3% black pigment) + | | | |
| | Composition 2-3 (SUNSHINE ®Northern | | | |
| | Lights.) | | | |
| H1 | Composition 1H (12.5% film-forming | 22.8 | 1.3 | 2 |
| | polymers, 3% black pigment) | | | |
| H2 | Composition 1H (12.5% film-forming | 45.7 | 18.5 | −7.7 |
| | polymers, 3% black pigment) + | | | |
| | Composition 2-2 (SUNSHINE ® Spectral Red) | | | |
| H3 | Composition 1H (12.5% film-forming | 65.8 | −25.8 | 7.6 |
| | polymers, 3% black pigment) + | | | |
| | Composition 2-3 (SUNSHINE ® Northern | | | |
| | Lights) | | | |

Conclusions:
The lightness of the first composition+the second composition was higher for A2 and A3 containing 25% of film-forming agent than for H2 and H3 containing 12.5% of film-forming agent. The glossiness was thus higher.
The red interference color of the nacre SUNSHINE® Spectral Red was more intense with the first composition at 25% of film-forming agents than with the first composition at 12.5% of film-forming agents (a=39.6 for sample A2 and a=18.5 for sample H2).
The green interference color of the nacre SUNSHINE® Northern Lights was more intense with the first composition at 50% of film-forming agents than with the first composition at 25% of film-forming agents (a=−29.4 for sample A3 and a=−25.8 for sample H3).

Impact of the Nature of the Film-Forming Agents:

Comparison of composition 1 containing different film-forming polymers, before and then after application of composition 2 containing the nacre SUNSHINE® Spectral Red or the nacre SUNSHINE® Northern Lights:

| | | | | | Composition 1A | | | Composition 1A | | |
|---|---|---|---|---|---|---|---|---|---|---|
| | | | | | (25% film-forming | | | (25% film-forming | | |
| | | Composition 1A | | | polymers, 3% pigment) + | | | polymers, 3% pigment) + | | |
| | | (25% film-forming | | | Composition 2-2 (SUNSHINE ® | | | Composition 2-3 (SUNSHINE ® | | |
| | | polymers, 3% black pigment) | | | Spectral Red) | | | Northern Lights) | | |
| | | L | a | b | L | a | b | L | a | b |
| 1 | Baycusan | 20.5 | 1.5 | 1.6 | 57.1 | 39.2 | 0.4 | 67.6 | −24.3 | −14 |
| | C1000 | | | | | | | | | |
| 2 | Baycusan | 21.2 | 1.5 | 1.8 | 58.2 | 34.5 | −2.1 | 60.2 | −22.9 | −9.8 |
| | 1004 | | | | | | | | | |
| 3 | Daitosol | 18.1 | 2.8 | 0.4 | 50.1 | 28.8 | 2.3 | 37.3 | −3.9 | −8.2 |
| | 4000 SJT | | | | | | | | | |
| 4 | Daitosol | 20 | 3.6 | 0.4 | 59.6 | 35.3 | −0.6 | 57.2 | −14.8 | −26.1 |
| | 5000 AD | | | | | | | | | |
| 5 | Daitosol | 23.1 | 3.4 | 2.4 | 46.6 | 20.4 | 3.9 | 35.4 | −0.9 | −6.2 |
| | 5000 PO | | | | | | | | | |
| 6 | Daitosol | 21.9 | 8.7 | −1.1 | 47.8 | 33.2 | 5.7 | 51.1 | −17.8 | −8.1 |
| | 5000 SJ | | | | | | | | | |
| 7 | Daitosol | 28 | 5.7 | −2.5 | 47.3 | 25.2 | −2.5 | 48.2 | −11.6 | −5.2 |
| | 5000 STY | | | | | | | | | |
| 8 | Daitosol | 24.5 | 0.8 | −3.8 | 64.3 | 47 | 13.1 | 63.5 | −21.1 | −28.4 |
| | 5500 GM | | | | | | | | | |
| 9 | Daitosol Plus | 25.4 | 1.3 | 6.4 | 31.6 | 9.3 | 0.5 | 29.4 | −0.6 | −2.9 |
| | MT-PE | | | | | | | | | |
| 10 | Koboguard | 20.4 | 2.5 | 1.6 | 51 | 28.5 | −0.6 | 47.5 | −12.2 | −8.9 |
| | 50AMP | | | | | | | | | |
| 11 | Syntran | 18.4 | 5.9 | 4.4 | 31 | 15.9 | 0.8 | 36.6 | 1 | −7.8 |
| | 5760CG | | | | | | | | | |
| 12 | syntran KL- | 19.9 | 2.9 | 3.7 | 26.7 | 12.6 | 3.1 | 25.5 | 0.4 | −2.5 |
| | 219CG | | | | | | | | | |
| 13 | syntran | 21.9 | 2.4 | 1.1 | 42.9 | 18.9 | −1.5 | 34.3 | −1.8 | −6.5 |
| | PC5778B | | | | | | | | | |
| 14 | Vinysol | 31.4 | 0.8 | 0.6 | 46.9 | 22.6 | −2.9 | 58.5 | −21.4 | −5.7 |
| | 2140L | | | | | | | | | |
| 15 | Worlée | 23.7 | 3 | −1.8 | 28.5 | 10.6 | −1.3 | 44.5 | −7.2 | −6.9 |
| | Micromer | | | | | | | | | |
| | C80/50 | | | | | | | | | |

Conclusions:
The lightness of composition 1+composition 2 was higher for composition Baycusan 01000, Baycusan 1004, Daitosol 5000 AD, and Daitosol 5500 GM. The glossiness was thus higher.
The red interference color of the nacre SUNSHINE@ Spectral Red was more intense with Baycusan C1000, Baycusan 1004, Daitosol 5000 AD, and Daitosol 5500 GM (higher a value).
The green interference color of the nacre SUNSHINE@ Northern Lights was more intense with Baycusan 01000, Baycusan 1004, Daitosol 5000 AD, and Daitosol 5500 GM (higher negative a value).
Baycusan 01000, Baycusan 1004, Daitosol 5000 AD, and Daitosol 5500 GM are thus the preferred film-forming agents for the invention.

1. Visual Evaluation by a Panel of Consumers

Panel: 11 women in the age range 20 years to 50 years evaluated:

A3 relative to H3;

A3 relative to F3; and

A3 relative to I.

For each pair, they were asked to compare the visual result and to indicate which had the higher glossiness and the higher color intensity according to them.

Here are the results of the study:
10 of the 11 panelists found that A3 had higher glossiness and higher color intensity tan H3, i.e. 89°/© of the panelists;
9 of the 11 panelists found that A3 had higher glossiness and higher color intensity than F3, i.e. 78% of the panelists; and
11 of the 11 panelists found that A3 had higher glossiness and higher color intensity than I, i.e. 100% of the panelists.

They were also asked to describe the visual effect obtained for A3. Here are the adjectives they used:

Metallic (×8), aluminized, colored aluminum foil, light or bright (×7), intense or bright color (×4), glossy (×7), glittery, and vibrant or vivid color (×3).

1. A skin cosmetic makeup set comprising:
a first composition to be applied to the skin so as to form at least one layer, called first layer; and
a second composition to be applied over all or a part of said first layer;
said first composition comprising:
an aqueous solvent;
at least one black pigment; and
at least one film-forming agent, the at least one film-forming agent representing in the range 10% to 45% by weight of the total weight of said first composition;
said second composition comprising at least one interference pigment, the interference pigment comprising a transparent substrate coated with at least one layer of coating material, said coating material being titanium dioxide (TiO₂).

2. A cosmetic set, wherein the lightness (L) of said first composition in the CIELAB color space is less than 27.5.

3. A cosmetic set according to claim 1, wherein the at least one film-forming agent of the first composition is selected from the group consisting of acetyltributylcitrate, acrylate polymer or their derivatives, acrylamide polymer or their derivatives, cellulose or its derivatives, chitosan or its derivatives, rosin, resin, gum, polyquaternium and mixtures thereof.

4. A cosmetic set according to claim 1, wherein the at least one film-forming agent of said first composition represents in the range 15% to 30% by weight of the total weight of said first composition.

5. A cosmetic set according to claim 1, wherein the black pigment of said first composition represents in the range 0.5% to 30% by weight of the total weight of said first composition.

6. A cosmetic set according to claim 1, wherein the interference pigment of said second composition comprises a transparent substrate selected from the group consisting of silica, mica, and glass.

7. A cosmetic set according to claim 1, wherein it is peelable.

8. A skin cosmetic makeup multilayer product comprising:
at least one first layer of said first composition as defined in claim 1; and
at least one second layer of said second composition as defined in claim 1, said second layer being applied over all or a part of said first layer.

9. A skin makeup kit comprising:
said first composition as defined in claim 1; and
said second composition as defined in claim 1.

10. A skin makeup method comprising:
applying at least one first layer of said first composition to the skin, said first composition being as defined in claim 1; and
then applying at least one second layer of the second composition over all or a part of said first layer, the second composition being as defined in claim 1.
